(19) United States
(12) Patent Application Publication
(10) Pub. No.: US 2026/0360542 A1
(43) Pub. Date: Nov. 12, 2026

(54) Detection System for Alzheimer's Disease Using Brain Structural and/or Functional Magnetic Resonance Image Processing and Machine Learning Techniques

(71) Applicant: Council of Scientific & Industrial Research, New Delhi (IN)

(72) Inventors: Saikat CHAKRABARTI, Kolkata (IN); Subhrangshu DAS, Kolkata (IN); Priyanka PANIGRAHI, Kolkata (IN)

(21) Appl. No.: 19/490,733

(22) PCT Filed: May 29, 2024
(86) PCT No.: PCT/IN2024/050632

§ 371 Date: Dec. 5, 2025

(30) Foreign Application Priority Data
Jun. 9, 2023 (IN) 202311039559

(51) Int. Cl. G16H 50/20; A61B 5/00; G06N 3/0464; G06V 10/764; G16H 50/70

(52) U.S. Cl. CPC G16H 50/20; A61B 5/4088; G06N 3/0464; G06V 10/764; G16H 50/70

(57) ABSTRACT

Early and accurate identification of dementia, such as Alzheimer's disease (AD) is of utmost importance. However, despite several decades of biomarker research, there is still no single diagnostic test or biomarker to definitively diagnose dementia and its underlying neurodegenerative disorder. A combination of structural MRI (fMRI) and functional MRI (fMRI) derived image analysis may provide the unique ability to capture the dynamic state of change in the degenerating brain. Hence, to capture the overall structural and functional anomalies of brain tissues caused by AD, an exhaustive combinatorial system has been developed using structural and/or functional MRI data followed by rigorous image processing and deep learning-based algorithms to diagnose AD and/or mild cognitive impairment (MCI) patients. Whole brain sMRI and fMRI slices are processed, and brain pixel-based intensity features are fed into a deep learning-based convoluted neural network (CNN) algorithm to calculate the AD/healthy probability of each MRI slice.

### FIELD OF THE INVENTION

The present invention relates to a detection system that utilizes image processing, machine learning, and statistical analysis to detect structural and functional changes that appear in the human brain during the early and late stages of Alzheimer's disease. In present invention the system is capable of processing and analyzing both brain structural MRI (sMRI) and functional MRI (fMRI) data where deep learning techniques such as convoluted neural network (CNN) algorithms were trained and tested to distinguish AD patients from normal cohorts.

The detection analysis package is embedded within a system comprising one computer processor unit, memory, and storage media to execute and store the programs and subsequent results thereon.

### BACKGROUND OF THE INVENTION

A large body of research has shown that imaging techniques are most promising technologies to improve subclinical and early diagnosis of dementia. Brain MRI analysis is one of the most active fields in the medical image analysis community. For the last couple of decades, a vast number of researches have come up with accurate extraction of quantitative measures from brain MRI data. These studies showed that there is scope to develop automatic classifier to identify AD or mild cognitive impairment (MCI) and apply a more quantitative approach both in dementia research and in clinical practice.

Use of different machine learning (ML) algorithms in analyzing bio-medical images is quite common since last decade. Different ML algorithms e.g. discrete wavelet transform (DWT), (Wulandari et al., 2018) principal component analysis (PCA) (Lama et al., 2017, Aruchamy et al., 2020) have been used to identify significant features and K-Means, Fuzzy C-Means (FCM) (Kar and Majumder, 2019) for clustering where Random Forest (RF) (Ezzati et al., 2019, Moore et al., 2019), K-nearest neighbor (KNN) (Borgohain et al., 2021, Acharya et al., 2019), and support vector machine (SVM) (Uysal and Ozturk, 2019, Long et al., 2017) have been used for classifying data. But, among all ML, artificial neural network (ANN) and convolutional neural network (CNN) (Pan et al., 2020, Lin et al., 2018, Islam and Zhang, 2018, Folego et al., 2020, and Bae et al., 2020) have been found to be widely used for identifying biomarkers and classifying bio-medical images. The implementation of CNN involves steps like finding convoluted layer followed by max pooling in a repeated way using open-source python library and modules such as Tensorflow, Keras, Theano. However, these systems require powerful graphics and high speed processor and take comparatively more time and effort to standardize and develop optimized models for better prediction accuracy.

In one of the previous works, (Das et al., 2021) the applicants of the current application showed morphological changes of corpus callosum (CC) caused by AD can be captured from sMRI data and further be used as two-dimensional (2D) features to feed into a multivariate pattern analysis using statistical machine learning technique, support vector machine (SVM), which distinguished AD and MCI patients from healthy ones with very high accuracy. Further, tools and techniques were developed for automatic identification followed by segmentation of corpus callosum (CC) using the publicly available Alzheimer's Disease Neuroimaging Initiative (ADNI) data from a large number of AD patient and healthy cohorts.

By reference to US Patent application number U.S. Pat. No. 11,151,717B2 discloses a method implementing image processing and machine learning techniques through utilization of structural MR images and PET image data for AD diagnosis. The system uses as inputs both functional positron emission tomography and structural magnetic resonance imaging data, reconstructs a model of the patient's cortex, uses machine learning techniques to generate probabilities for mild cognitive impairments for local cortical regions, uses machine learning techniques to fuse the local diagnoses to generate a global diagnosis based on each imaging modality, then uses machine learning techniques to fuse the modality-specific global diagnoses to generate a final global diagnosis.

The methodology of the present invention is different from the above-mentioned invention. The present invention utilizes whole-brain MR imaging (MRI & fMRI) to distinguish AD patients from the normal cohorts. The present invention does not require any supervision. It uses combinations of deep learning (CNN) protocols/models to distinguish AD patients from normal cohorts. The present invention is applicable to varying degrees of AD, i.e., from mild to moderate and up to severe Alzheimer's disease.

By reference to CN Patent application number CN112767374A relates to an MRI-based Alzheimer disease focus region semantic segmentation algorithm, and belongs to the field of computer vision and medical image processing. This method utilizes a semantic segmentation algorithm for a focus region of Alzheimer's disease based on MRI and takes a general semantic segmentation network as a basic frame, strengthens feature expression and attention to salient features, and extracts features aiming at focus regions with different scales so as to more accurately determine the position of each focus region.

This method uses U-net network framework. The limitations of U-Net include low accuracy, low precision, and a cumbersome detection process, which can affect training and accuracy. The present invention is much more exhaustive and sophisticated. Combination structural and functional MRI data processing and analysis aid the present invention in obtaining higher efficacy. It is a reasonably rapid method of detection of AD which gives results within 15-30 minutes. The present invention produces excellent efficacy with 90% or more accuracy for both testing and validation cohort-based benchmarking analyses.

Effective utilization of both brain fMRI and sMRI data is relatively less explored. A combination of both functional and/or structural anomalies, if captured, can provide early but probable persistent signs of developing dementia. Most of the existing software/programs are not readily available, not technically amenable, and require equivalent multi-component third-party software packages and dependencies. A single platform that could process raw sMRI and fMRI data and extract features to identify structural and functional anomalies and subsequently utilize those features to train powerful machine learning systems to detect/diagnose AD is missing. In agreement with previous observations, it could be extrapolated that although many algorithms could differentiate AD from normal samples with satisfying accuracy, distinguishing the MCI from normal remains a major challenge.

### OBJECTIVE OF THE INVENTION

The main objective of the current invention is to detect and diagnose AD and/or MCI patients through computer-aided MR image processing and subsequent analysis using a deep learning based classification and prediction system comprising of trained models generated from large scale AD patients and healthy control cohorts via exhaustive feature extraction and training-testing experiments, instructions/package to process query MRI data, testing of query data with respect to the trained models, prediction module to portray the probable diagnosis of the query data embedded within a hardware system comprising one standard computer processor unit, memory and storage media to execute and store the programs and subsequent results thereon. Another objective of the invention is to utilize both sMRI and fMRI data capable of capturing the structural and functional anomalies that may appear during early and late stages of the AD/MCI disease progression. Implementation of this embedded system to be synced with the clinically used MRI/fMRI machines

In another objective the present invention processes whole brain slices from axial, sagittal, and coronal views of the given sMRI and/or fMRI data and further extracts features to feed into a deep learning-based CNN module to calculate the AD/healthy probability of each slice.

Another objective of the invention is to implement statistical analysis to diagnose the AD status of the given MRI sample based on the AD probability computed from three different views of the sMRI and/or fMRI data.

Another objective of the invention is to develop the AD detection analysis system comprising one computer processor unit, memory and storage media to execute and store the programs and subsequent results thereon.

Another objective of the invention is to provide a graphical user interface (GUI) based user-interactive platform embedded within the detection system for prediction of the AD status of the query sample sMRI/fMRI data.

### SUMMARY OF THE INVENTION

In one aspect, the present relates to a system (300) for detecting neurodegenerative disorder comprising;
i) at least one computer processor unit (330);
ii) at least one memory media (340);
iii) one graphical user interface-based display unit (350); and
iv) one storage medium (360).
wherein the system analysing and predicting brain structural MRI slices data and functional MRI slices data followed by implementation, customization, and standardization of Convoluted Neural Network algorithms.

In another aspect, the present relates to a method (200) for predicting the functional MRI by the system of claim 1, wherein the method comprising the steps of;
(a) collecting (210) functional MRI data from Alzheimer's Disease Neuroimaging Initiative (ADNI) database;
(b) converting the functional MRI data of step (a) using data pre-processing module (220) into 2D images for the ease of handling the images and further pre-processing of the images;
(c) extracting the 2D images of step (b) in three different views, i.e., axial, coronal, and sagittal, respectively;
(d) extracting the pixel values of the 2D images of step (c) using file preparation module (230);
(e) classifying the pixel images obtained in step (d) through Convoluted Neural Network module (240) using training-testing-validation protocol along with standardization of parameters and hyper-parameters followed by detailed standardization of Convoluted Neural Network module;
(f) combining all standardization of step (b) to (e) by standalone hardware system (280) comprising one standard computer processor unit, memory, and storage media of claim 1 to predict the disease status in the prediction module (290).

In one another aspect, the present invention relates to method (200) for predicting the functional MRI by the system of claim 1, wherein the method comprising the steps of;
(a) collecting (210) functional MRI data from Alzheimer's Disease Neuroimaging Initiative (ADNI) database;
(b) converting the functional MRI data of step (a) using data pre-processing module (220) into 2D images for the ease of handling the images and further pre-processing of the images;
(c) extracting the 2D images of step (b) in three different views, i.e., axial, coronal, and sagittal, respectively;
(d) extracting the pixel values of the 2D images of step (c) using file preparation module (230);
(e) classifying the pixel images obtained in step (d) through Convoluted Neural Network module (240) using training-testing-validation protocol along with standardization of parameters and hyper-parameters followed by detailed standardization of Convoluted Neural Network module;
(f) combining all standardization of step (b) to (e) by standalone hardware system (280) comprising one standard computer processor unit, memory, and storage media of claim 1 to predict the disease status in the prediction module (290).

### ABBREVIATIONS

AD: Alzheimer's disease
MCI: Mild cognitive impairment
MRI: Magnetic Resonance Imaging
sMRI: Structural Magnetic Resonance Imaging
fMRI: Functional Magnetic Resonance Imaging
CNN: Convoluted Neural Network
ADNI: Alzheimer's Disease Neuroimaging Initiative

### BRIEF DESCRIPTION OF DRAWINGS

FIG. 1: Schematic overview of the approach and methods implemented in the system for sMRI-based analysis and prediction, as per an embodiment herein.

FIG. 2: Schematic overview of the approach and methods implemented in the system for fMRI-based analysis and prediction, as per an embodiment herein.

FIG. 3: Schematic diagram of the various components of the apparatus/device for detection of AD using sMRI/fMRI data as input.

FIG. 4: Schematic representation of the detection device along with various input and output option.

FIG. 5: Flowchart of the prediction module where each sMRI and/or fMRI slice from three views are processed using all training models.

FIG. 6: Flowchart of the prediction module where all sMRI and/or fMRI slices from three views are processed using each training model.

### DETAILED DESCRIPTION OF THE INVENTION

While the disclosure has been disclosed with reference to certain embodiments, it will be understood by those skilled in the art that various changes may be made, and equivalents may be substituted without departing from the scope of the invention. In addition, many modifications may be made to adapt to a particular situation or material to the teachings of the invention without departing from its scope.

In one embodiment the present invention aims to detect and diagnose AD and/or MCI patients through a computer-aided MR image processing and subsequent analysis using a deep learning based classification and prediction system comprising of trained models generated from large scale AD patients and healthy control cohorts via exhaustive feature extraction and training-testing experiments, instructions/package to process query MRI data, testing of query data with respect to the trained models, prediction module to portray the probable diagnosis of the query data embedded within a hardware system comprising one standard computer processor unit, memory and storage media to execute and store the programs and subsequent results thereon. In one embodiment, the system is composed of two independent yet complimentary diagnosing approaches where one part is capable of processing and analyzing brain structural MRI (sMRI) data followed by implementation, customization, and standardization of convoluted neural network (CNN) algorithms to distinguish AD patients from normal healthy cohorts.

In another embodiment, the present invention provides a description of AD detection using sMRI data (100). Data collection module (110) depicts collection of sMRI data from the Alzheimer's Disease Neuroimaging Initiative (ADNI) database consisting of 553 healthy and 1156 AD patients (severe AD: 53, moderate AD: 237, and mild AD: 866) followed by data pre-processing module (120), which converted all 3D data (NIFTI format) of the brain structural MR images into 2D slices for the ease of handling the images and further pre-processing of the images. 2D image slices were extracted in three different views, i.e., axial, coronal, and sagittal, respectively.

In another embodiment, the feature extraction and file preparation module (130) extract the pixel values of the images and uses them as features to perform machine learning-based classification.

Classification via k-fold training-testing and subsequent validation of the models using convoluted neural network (CNN) module (140) employs a subset of the AD and healthy cohorts for training-testing exercise, and the remaining samples were kept separated for a validation dataset. The following sections depict the model architecture and summary properties (150).

Model architecture: A sequential model was used for this exercise. The sequential model is a linear stack of layers. It can be first initialized and then layers can be added using various methods. Conv2D is a 2-dimensional convolution layer, which performs spatial convolution over images. The parameters used are Filters, Kernel size, Activation, Kernel initializer, and Input shape. MaxPooling2D is a max pooling operation for spatial data. The parameters used here are Pool size (2,2). Conv2D with 64 filters, 3×3 kernel size and Relu activation. MaxPooling2D with pool size (2,2). Conv2D with 128 filters, 3×3 kernel size and Relu activation. Flatten and dense layers were used with default parameters, Activation function (dense layer): Relu and Activation function (dense layer): Sigmoid.

Compilation of Model: The model was compiled using parameters such as Loss function, Adam optimizer and Performance metrics (e,g., accuracy and/or F1 score).

Model Summary: Keras was used which appends an extra dimension for processing multiple batches to train multiple images in every step of a single Epoch. Convolution was performed using various size filters via Conv2D and Maxpooling2D. The flatten layer takes all the pixels along all channels and creates a 1D vector (not considering batch size). Two dense layers were added having 128 and 2 units, respectively.

Standardization of hyper-parameters like epoch and batch-size: Hyper-parameters like epoch and batch-size were standardized to train the dataset. The batch-size is a hyper-parameter of gradient descent that controls the number of training samples to work through before the model's internal parameters are updated. The number of epochs is a hyper-parameter of gradient descent that controls the number of complete passes through the training dataset. Multiple combinations of epoch and batch-size were used for training. Table 1 provides performance values of training-testing and validation cohorts both in terms of slice-wise and patient predictions for the using AD vs. Healthy models.

TABLE 1
Performance of All AD vs Healthy models using sMRI data

| Brain | Train-Test | Validation |
|---|---|---|
| Anatomical | F1 Score (Slice-wise | F1 Score (Slice-wise |
| View | prediction) | prediction) |
| Axial | 0.89 | 0.89 |
| Coronal | 0.90 | 0.90 |
| Sagittal | 0.91 | 0.91 |

In another embodiment of the system, fMRI data was utilized for AD detection (200). Data collection module (210) depicts a collection of fMRI data from the Alzheimer's Disease Neuroimaging Initiative (ADNI) database consisting of 170 healthy, 114 AD, and 170 MCI patients followed by data pre-processing module (220), which converted all 3D data (NIFTI format) of the brain functional MR images into 2D slices for the ease of handling the images and further pre-processing of the images. 2D image slices were extracted in three different views, i.e., axial, coronal, and sagittal, respectively.

In another embodiment, the feature extraction and file preparation module (230) extracts the pixel values of the images and use them as features to perform machine learning-based classification.

Classification via k-fold training-testing and subsequent validation of the models using convoluted neural network (CNN) module (240) employs a subset of the AD and healthy cohorts for training-testing exercise and remaining samples were kept separated for a validation dataset. Following sections depict the model architecture and summary properties (250).

Model architecture: Sequential model was used for this exercise using Conv2D 2-dimensional convolution layers. 3 Conv2D layers with Relu activation and two times MaxPooling were performed. Flatten and 2 dense layers were used with default parameters, Activation function (dense layer): Relu and Activation function (dense layer): Sigmoid.

Compilation of Model: The model was compiled using parameters such as Loss function, Adam optimizer and Performance metrics (e,g., accuracy and/or F1 score).

Model Summary: Keras was used which appends an extra dimension for processing multiple batches to train multiple images in every step of a single Epoch. Convolution was performed using various size filters via Conv2D and Maxpooling2D.

Rigorous standardization of the hyper-parameter combinations was done in order to obtain optimum combinations of epoch and batch-size for training. Exhaustive benchmarking using testing and separate validation datasets was carried out. Table 2 provides performance values of training-testing and validation cohorts both in terms of slice-wise and patient-wise predictions for all AD vs. Healthy models.

TABLE 2
Performance of AD vs Healthy models using fMRI data

| | |
|---|---|
| Train-Test | Validation |

| | | | | |
|---|---|---|---|---|
| Brain | Slice- | Patient- | Slice- | Patient- |
| Anatomical | wise | wise | wise | wise |

| | | | |
|---|---|---|---|
| View | Models | F1 Score | F1 Score |

| | | | | | |
|---|---|---|---|---|---|
| Axial | Top 10 | 0.80 | 0.98 | 0.83 | 0.98 |
| Coronal | Top 10 | 0.77 | 0.92 | 0.75 | 0.90 |
| Sagittal | Top 10 | 0.75 | 0.91 | 0.77 | 0.91 |

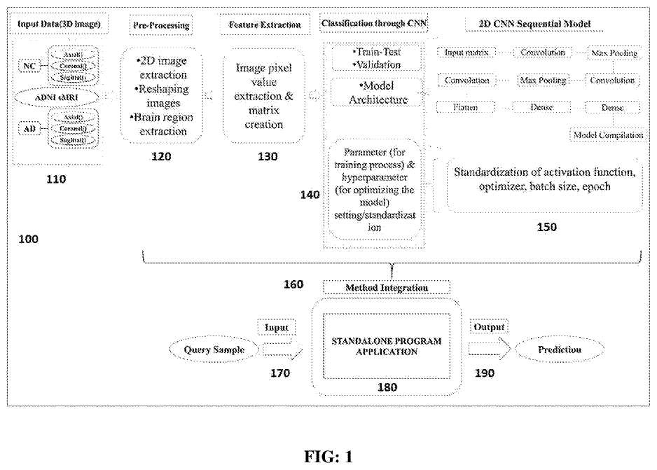

In another embodiment, FIG. 1 shows an overview of the method 100 for implemented in the system for sMRI based analysis and prediction. The process begins with structural MRI image collection and data processing (110) followed by image preprocessing and feature extraction steps (120 and 130). Classification CNN module using training-testing-validation protocol along with standardization of parameters and hyper-parameters (140) whereas detailed standardization of the CNN modules are done in 150. Step 160 combines all standardized and trained models via developing a standalone hardware system (180) comprising one standard computer processor unit, memory and storage media to execute and store the programs and subsequent results thereon, which could accept input data (170) and provide disease status outcome prediction in the prediction module (190).

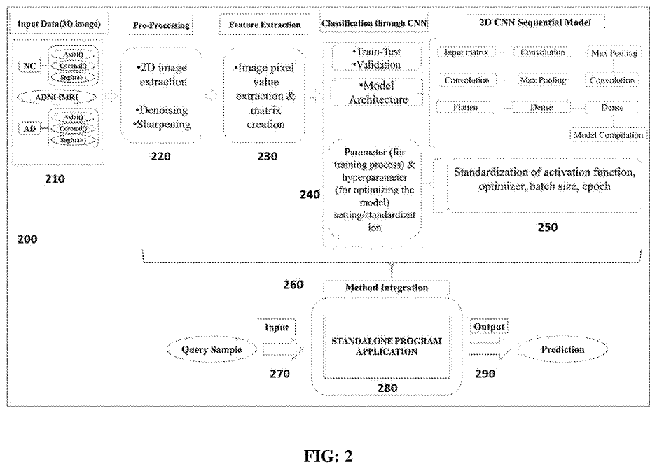

In another embodiment, FIG. 2 shows an overview of the method 200 implemented in the system for fMRI-based analysis and prediction. The process begins with structural MRI image collection and data processing (210) followed by image preprocessing and feature extraction steps (220 and 230). Classification of CNN module using training-testing-validation protocol along with standardization of parameters and hyper-parameters (240), whereas detailed standardization of the CNN modules is done in 250. Step 260 combines all standardized and trained models via developing a standalone hardware system (280) comprising one standard computer processor unit, memory, and storage media to execute and store the programs and subsequent results thereon, which could accept input data (270) and provide disease status outcome prediction in the prediction module (290).

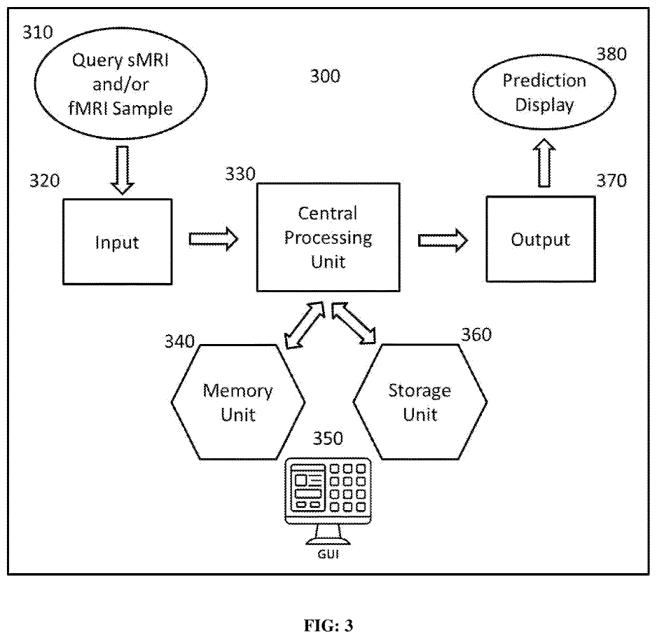

In another embodiment (FIG. 3, 300), the invention provides a computer-aided, whole-brain structural/functional MR image analysis-based AD diagnostic system. The system comprising: i) At least one computer processor for executing the instructions therein (330); ii) At least one memory unit (340), one GUI based display unit (350), one computer-readable storage medium (360) to store the instructions along with pre-compiled trained models for live prediction and/or detection; iii) Pre-computed, standardized, and trained models and dataset generated from brain sMRI/fMRI data and associated instructions for utilization of these models in detection of MR slices with probable AD/healthy signatures using CNN based deep learning methods (360), iv) A platform/system for input, query, brain sMRI/fMRI data retrieval and real-time brain MR image pre-processing and processing (320), v) Programmable instructions to extract features from the query sample MR images (330, 340 and 360), vi) Programmable instructions compare and match the query brain MR image features with pre-existing, pre-computed, standardized, and trained models via invoking CNN methodologies (330, 340, and 360), vii) Programmable instructions to execute statistical analysis and predict the AD status of the query sample (330, 340, 350, and 360), viii) Algorithmic and programmable instructions to execute statistical analysis and predict the healthy or AD status of the query sample (350, 370 and 380).

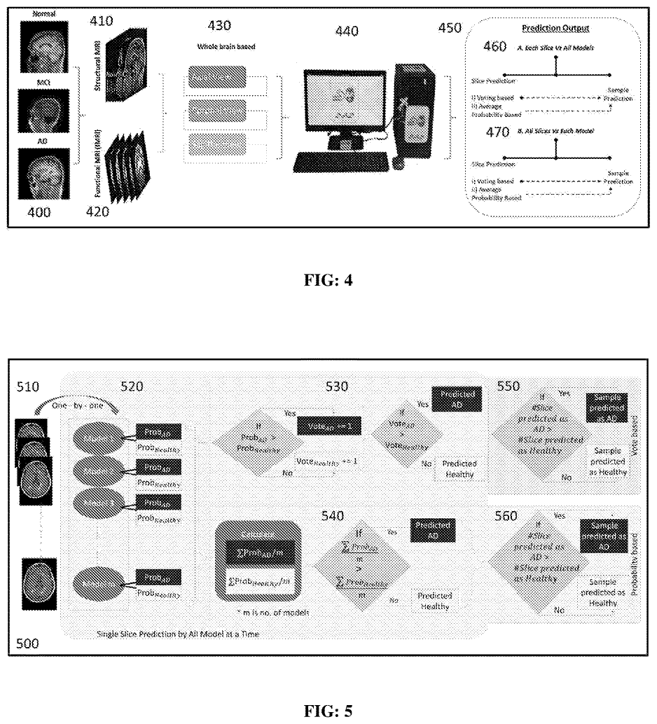

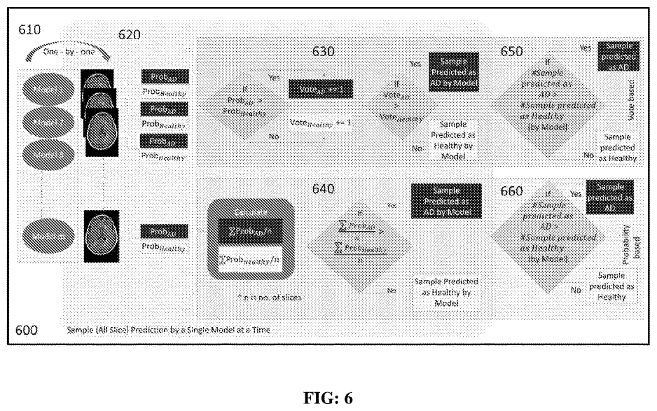

In another embodiment (FIG. 4, 400), the invention provides the prototype of the AD detection device along with its input (410, 420, 430) and output (450, 460, and 470) options. Sample MRI images from AD/MCI and/or healthy individuals are shown as examples. MRI slices (410 and 420) from axial, coronal, and sagittal views can be used as input separately. Various image processing modules embedded within the device perform pre-processing, processing, feature extraction, matrix generation, etc, and subsequently, the machine learning modules empowered by the CNN algorithm compare the input image slices against the pre-developed trained models derived from the MRI data from a large number of AD/MCI and healthy individuals. The prediction output module (450) contains two independent strategies (460 and 470) for slice and sample prediction. Details of each strategy are presented in FIG. 5 and FIGS. 6 (500 and 600), respectively.

In another embodiment (FIG. 5, 500), the invention provides a detailed flowchart of the steps incorporated within the prediction module where each sMRI and/or fMRI slice from three views is processed using all training models. Here, AD/MCI/healthy like probability of the processed data extracted from each MRI slice (510) is estimated via classification and prediction against all the training models (520). 530 and 540 provide two submodules where voting and average probability-based prediction of each slice is performed. Sub-modules 550 and 560 deal with AD/MCI/healthy status prediction for the whole sample for three different views after consolidating prediction results from each slice.

In another embodiment (FIG. 6, 600), the invention presents a detailed flowchart of the steps incorporated within the prediction module where all sMRI and/or fMRI slices from three views are processed using each training model at a time. Here, the whole sample (all slices) from each view is classified against each of the training model. 630 and 640 provide two submodules where voting and average probability-based prediction of each model is performed. Sub-modules 650 and 660 deal with AD/MCI/healthy status prediction for the whole sample for three different views after consolidating prediction results from each model

### ADVANTAGES

1. The present invention aims to detect and diagnose AD and/or MCI patients through a computer-aided MR image processing and subsequent analysis using a deep learning-based classification and prediction system comprising trained models generated from large-scale AD patients and healthy control cohorts via exhaustive feature extraction and training-testing experiments, instructions/package to process query MRI data, testing of query data with respect to the trained models, prediction module to portray the probable diagnosis of the query data embedded within a hardware system comprising one standard computer processor unit, memory and storage media to execute and store the programs and subsequent results thereon.
2. The system is composed of two independent yet complimentary diagnosing approaches where one part is capable of processing and analyzing brain structural MRI (sMRI) data followed by implementation, customization, and standardization of convoluted neural network (CNN) algorithms to distinguish AD patients from normal healthy cohorts.
3. In the other part, this package processes, analyzes, and predicts AD/MCI status using functional MRI (fMRI) slices. In this case, whole brain slices from axial, sagittal, and coronal views are processed, and brain pixel-based intensity features are fed into a deep learning-based CNN module to calculate the AD/healthy probability of each slice. Further, statistical analysis is employed to diagnose the AD status based on the AD probability computed from three different views.
4. This detection system uniquely offers to detect AD/MCI using both sMRI and fMRI data capable of capturing the structural and functional anomalies that may appear during early and late stages of the disease progression. Hence, this embedded system could easily be synced with the clinically used MRI/fMRI machines. This detection system can aid clinical diagnostics of AD and AD like symptoms in an efficient manner.
5. This detection system uniquely offers AD/MCI/healthy prediction status of each MRI slice from three different views (e.g., axial, coronal, and sagittal, respectively).
6. This detection system provides AD/MCI/healthy status prediction for the whole sample for three different views after consolidating prediction results from each slice.
7. This detection system uniquely offers multiple strategies based on different yet complimentary statistical analyses of the prediction. In one embodiment, the invention incorporates a prediction module where each sMRI and/or fMRI slice from three views is processed using all training models. In another embodiment, the invention presents a prediction module where all sMRI and/or fMRI slices from three views are processed using each training model at a time.
8. This detection system uniquely offers two submodules where voting and average probability-based prediction of each slice are performed.
9. This detection system contains highly enriched sMRI training models consisting of 15 All AD vs Healthy, 15 Mild vs Healthy, 15 Moderate vs Healthy, and 15 Severe vs Healthy models. All AD vs Healthy models contain data from 354 AD and 354 healthy samples, containing 84940 slices in each view (axial, coronal, and sagittal, respectively). All Mild vs Healthy models contain data from 354 mild and 354 healthy samples, containing 135936 slices in axial and coronal view and 103368 slices in sagittal view. All Moderate vs Healthy models contain data from 151 moderate and 151 healthy samples, containing 57984 slices in axial and coronal view and 44092 slices in sagittal view. All Severe vs Healthy models contain data from 34 severe and 34 healthy samples, containing 13056 slices in axial and coronal view and 9928 slices in sagittal view.
10. This detection system contains highly enriched fMRI training models consisting of 300 AD vs Healthy and 300 MCI vs Healthy models. In each axial model, 73 and 108 demented and healthy samples containing 3504 and 5184 slices are present. In each coronal model, 73 and 108 demented and healthy samples containing 4672 and 6912 slices are present. In each sagittal model, 73 and 108 demented and healthy samples containing 4672 and 6912 slices are present.
11. Prediction accuracies of sMRI models are excellent as testified through exhaustive benchmarking using separate test and validation datasets.
12. Prediction accuracies of fMRI models are excellent as testified through exhaustive benchmarking using separate test and validation datasets.
13. This detection system contains a graphical user interface (GUI) based user-interactive platform embedded within the detection system for prediction of the AD status of the query sample sMRI/fMRI data.
14. This detection system executes prediction for a given query MRI samples in a relatively faster time scale (within 25-30 minutes).
15. This detection system could easily be synced with the clinically used MRI/fMRI machines. This detection system can aid clinical diagnostics of AD and AD like symptoms in an efficient manner.

1. A system for detecting neurodegenerative disorder comprising:
i) at least one computer processor unit;
ii) at least one memory media;
iii) one graphical user interface-based display unit; and
iv) one storage medium,
wherein the system analyzes and predicts brain structural MRI slices data and functional MRI slices data;
wherein the system comprises trained models generated from Alzheimer's disease (AD) patients and healthy cohorts to process query MRI data followed by testing of query data using the trained models providing prediction module to diagnose the query data;
wherein the prediction module is embedded in said system; and
wherein the brain structural MRI slices data and functional MRI slices data are selected from axial, sagittal, and coronal views.

2. The system as claimed in claim 1, wherein the neurodegenerative disorder is selected from Alzheimer's disease and mild cognitive impairment.

3. A method for predicting the structural MRI by the system of claim 1, wherein the method comprises the steps of:
(a) collecting structural MRI data from Alzheimer's Disease Neuroimaging Initiative (ADNI) database;
(b) converting the structural MRI data of step (a) using data pre-processing module into 2D images for the ease of handling the images and further pre-processing of the images;
(c) extracting the 2D images of step (b) in three different views, i.e., axial, coronal, and sagittal, respectively;
(d) extracting the pixel values of the 2D images of step (c) using file preparation module;
(e) classifying the pixel images obtained in step (d) through Convoluted Neural Network module using training-testing-validation protocol along with standardization of parameters and hyper-parameters followed by detailed standardization of Convoluted Neural Network module; and
(f) combining all standardization of step (b) to (e) by standalone hardware system comprising one standard computer processor unit, memory and storage media of claim 1 to predict the disease status in the prediction module.

4. A method for predicting the functional MRI by the system of claim 1, wherein the method comprises the steps of:
(a) collecting functional MRI data from Alzheimer's Disease Neuroimaging Initiative (ADNI) database;
(b) converting the functional MRI data of step (a) using data pre-processing module into 2D images for the ease of handling the images and further pre-processing of the images;
(c) extracting the 2D images of step (b) in three different views, i.e., axial, coronal, and sagittal, respectively;
(d) extracting the pixel values of the 2D images of step (c) using file preparation module;
(e) classifying the pixel images obtained in step (d) through Convoluted Neural Network module using training-testing-validation protocol along with standardization of parameters and hyper-parameters followed by detailed standardization of Convoluted Neural Network module; and
(f) combining all standardization of step (b) to (e) by standalone hardware system comprising one standard computer processor unit, memory, and storage media of claim 1 to predict the disease status in the prediction module.

5. The method as claimed in claim 4 wherein the Alzheimer's Disease Neuroimaging Initiative (ADNI) database comprises the data of healthy individuals, Alzheimer's Disease and mild cognitive impairment patients.

6. The method as claimed in claim 4, wherein the method includes severe, moderate, and mild Alzheimer's Disease detection.
